(19) United States
(12) Patent Application Publication
(10) Pub. No.: US 2025/0041513 A1
(43) Pub. Date: Feb. 6, 2025

(54) CABLE DRIVE SYRINGE PUMP SYSTEM

(71) Applicant: Medone Surgical, Inc., Sarasota, FL (US)

(72) Inventors: Bruce Beckstein, Sarasota, FL (US); Segundo Rodriguez, Sarasota, FL (US); Erik S. Brown, Sarasota, FL (US)

(21) Appl. No.: 18/718,197

(22) PCT Filed: Dec. 8, 2022
(86) PCT No.: PCT/US2022/081144

§ 371 Date: Jun. 10, 2024

Related U.S. Application Data
(60) Provisional application No. 63/288,395, filed on Dec. 10, 2021.

(51) Int. Cl. A61M 5/145

(52) U.S. Cl. CPC A61M 5/1452; A61M 2205/33

(57) ABSTRACT

Embodiments herein disclose a cable driven injection system provided with a syringe into which a fluid is filled and from which the fluid is discharged to an injection site. A stopper is coupled to and is slidable within an interior of the syringe. In addition, an actuator, such as an electric motor, may be configured to control movement of the stopper within the syringe to deliver a highly accurate dosage of fluid at a highly precise flow rate. The syringe is located remote from the actuator. A drive cable that includes a flexible inner wire and a flexible outer sleeve enclosing the flexible inner wire connects the actuator and the syringe such that activation of the actuator causes the flexible inner wire to move the stopper within the syringe. Some embodiments may include a controller and a software program configured to control the administration of fluid according to predetermined conditions.

### CROSS REFERENCE TO RELATED APPLICATIONS

This application claims the benefit of priority to U.S. Provisional Application 63/288,395, titled Cable Drive Syringe Pump System filed on Dec. 10, 2021, the entire contents of which are hereby incorporated by reference.

### TECHNICAL FIELD

The present disclosure relates generally to an injection system, and more particularly, to an injection system in which a syringe is connected to an actuator assembly via a drive cable.

### BACKGROUND

Injection devices are well known in the medical field for delivering medication. These injection devices typically include syringes for the injection and/or aspiration of fluids or other materials. Often syringes are coupled to a syringe pump to control the discharge of a particular volume of fluid from the syringe. In some uses, the syringe includes a needle tip that is directly inserted into an injection site for injection of the fluid. Alternately, the syringe may be connected to a tubing that is attached at the injection site, typically via an intra venous line (IV).

Some injection devices may include a syringe pump to operate the syringe to improve the control and precision of the volume of fluid that is discharged. The pump in these devices may be coupled to an actuator to move a plunger or stopper within the syringe and push the fluid or other material through the tubing of the syringe and into the injection site. These systems attempt to control the plunger at a predetermined rate to deliver the fluid according to a prescribed dose requirement. This type of injection of fluid is important for intravenous drug administration including for pain medications, antibiotics, cancer fighting drugs, and the like where precise control is critical. For example, many ophthalmic drug therapies require precise control of the delivered dose and flow rate of medication.

However, developed injection devices with these types of syringe pumps and actuators are bulky and often difficult to manipulate due to the design of the combined syringe and actuator. Many are not capable of delivering hyper accurate doses either. Some of these systems also require the user to manually operate the system to actuate the syringe which increases the risk of excessive movement during delicate surgical procedures. The developed systems are also not ergonomic for some surgical applications thus contributing to hand fatigue during use. In other developed systems, the motor of the actuator causes vibration during operation increasing the risk of error during administration of the fluid. For example, the over delivery of injectable material and the under delivery of injectable material.

Accordingly, there is a need to develop a syringe pump system that ensures for precise control of the fluid administration without requiring manual manipulation of an actuator.

The above information disclosed in this Background section is only for enhancement of understanding of the background of the disclosure and therefore it may contain information that does not form the prior art that is already known to a person of ordinary skill in the art.

### SUMMARY

The techniques of this disclosure generally relate to an apparatus and method for delivering a precise quantity of a therapeutic material at a precise flow rate via a cable driven syringe system.

According to various embodiments, the present disclosure provides an injection system that includes a syringe into which a fluid is filled and from which the fluid is discharged to an injection site. A stopper is coupled to the syringe and is slidable within an interior of the syringe. In addition, an actuator controls the stopper within the syringe which is located remote from the actuator. A drive cable connects the actuator and the syringe. The drive cable includes a flexible inner wire and a flexible outer sleeve that encloses the flexible inner wire.

Further, a first end of the flexible inner wire is connected to the stopper and a second end of the flexible inner wire is releasably connected to the actuator. A minimal gap is formed between the flexible inner wire and the flexible outer sleeve. This minimal gap may be about 0.001 to 0.002 inches. In various embodiments, the syringe includes a distal end which is configured to connect to a cannula tip through which the fluid may be discharged into the injection site. The syringe may be pre-filled with the fluid.

In some embodiments, the flexible outer sleeve includes a reinforcing wire braid that eliminates axial and lateral expansion of the flexible outer sleeve to improve accuracy of power transmission. The flexible outer sleeve is made of a polyimide material and the flexible inner wire is made of a nitinol material and has a thickness of about 0.025 to 0.027 inches. Additionally, an inner lumen of the flexible outer sleeve may include a polytetrafluoroethylene (PTFE) layer that reduces friction between the flexible inner wire and the flexible outer sleeve during movement. In some embodiments, the drive cable may be about 24 to 36 inches in length. In other embodiments, the drive cable may be about six feet in length.

In addition, the actuator is connected to a controller configured to execute an operation program of the injection system. The actuator is configured to move forward and backwards in response to a signal of the controller to push and pull the flexible inner wire and move the stopper within the syringe. The operation program may include a volume and a flow rate of the fluid to be discharged to the injection site. In some embodiments, the actuator is actuated by a foot pedal or button located remote from the syringe.

According to another embodiment, the present disclosure provides a method of injection a fluid to an injection site. The method may include connecting a syringe to a drive cable, the drive cable including a flexible inner wire and a flexible outer sleeve that encloses the flexible inner wire. The method then includes connecting the drive cable to an actuator connected to a controller and injecting a cannula tip connected to the syringe into the injection site. The controller then receives an operation program, and the actuator is actuated to activate a stopper coupled to and slidable within an interior of the syringe based on the operation program to discharge a fluid stored in the syringe into the injection site. The actuation of the actuator causes the actuator to move forward and backwards to push and pull the flexible inner wire and move the stopper within the syringe.

In some embodiments, prior to injecting the cannula tip connected to the syringe into the injection site, the method includes operating the actuator to advance the stopper to purge air bubbles within the syringe and/or the cannula tip connected to the syringe. The discharge of the fluid may be stopped automatically in response to determining that the operation program is completed. In some embodiments, the discharge of the fluid is stopped when the volume of the fluid reaches a predetermined threshold. In some embodiments, the discharge of the fluid is stopped and restarted manually.

A cable driven injection system is disclosed. The system may include a syringe including a stopper disposed within an interior cavity thereof that is configured to receive a fluid and discharge the fluid at a surgical site. The system may include an actuator assembly including an electric motor and a rotatable shaft configured to linearly translate a block in a longitudinal direction, and a drive wire coupled to the block and the stopper that is configured to move forward upon activation of the actuator assembly thereby moving the stopper forward and causing the syringe to discharge the fluid. The system may include a control box having a computing system including at least one controller and a non-transitory memory storage containing an operation program comprising computer executable code, and the operation program may contain target volume information for the discharge of the fluid. In various embodiments, the controller may be configured to supply a target power to the electric motor such that a volume of fluid discharged from the syringe corresponds to the target volume information of the operation program.

In various embodiments, upon supplying the target power to the electric motor, a measured amount of fluid discharged from the syringe is within +/−3 microliters or less of the target volume.

In various embodiments, the operation program further contains flow rate information for the discharge of the fluid, and the controller may be further configured to supply the target power to the electric motor such that a flow rate of fluid being discharged from the syringe corresponds to the flow rate information of the operation program. In various embodiments, the drive cable includes: a flexible inner wire; and a flexible outer sleeve enclosing the flexible inner wire.

In various embodiments, the operation program may be user customizable via a user interface. In various embodiments, the operation program further includes target volume information for a plurality of different fluids. In various embodiments, the operation program further includes target flow rate information for a plurality of different fluids. In various embodiments, the system may include an occlusion sensor configured to detect if the syringe is occluded; and the operation program may be further configured to cause an occlusion notification if the occlusion sensor indicates the syringe is occluded. In various embodiments, a flow rate sensor may be included that is configured to detect a volume and flow rate of fluid being dispensed from the syringe; and the operation program may be further configured to cause a volume notification and a flow rate notification to be displayed by a display in electrical communication with the computing system.

Notably, the present disclosure is not limited to the combination of the device elements as listed above and may be assembled in any combination of the elements as described herein. Other aspect of the disclosure are disclosed infra.

### BRIEF DESCRIPTION OF DRAWINGS

The above and other features of the present disclosure will now be described in detail with reference to certain embodiments thereof illustrated in the accompanying drawings which are given herein by way of illustration only, and thus are not limitative of the present disclosure, and wherein:

FIG. 1A shows a cable driven syringe pump embodiment;

FIG. 1B shows a control box embodiment;

FIG. 2A shows a first perspective view of a cable driven syringe pump system;

FIG. 2B shows a second perspective view of the cable driven syringe pump system;

FIG. 3A shows a detailed view of various internal components of the control box;

FIG. 3B shows a top-down view of some parts of FIG. 3A;

FIG. 4A shows an actuation assembly for the cable driven syringe pump system;

FIG. 4B shows a partial parts view of the actuation assembly of FIG. 4A with some parts removed for ease of understanding;

FIG. 4C shows an exploded parts view of the actuation assembly of FIG. 4A with some parts removed for ease of understanding;

FIG. 5A shows a side view of the cable driven syringe pump system;

FIG. 5B shows a cross section view from the perspective shown in FIG. 5A;

FIG. 6 shows a cross section view of a portion of the actuation assembly;

FIG. 7 shows the connection of the syringe to the control box;

FIG. 8A shows another side view of the cable driven syringe pump system;

FIG. 8B shows a cross section view from the perspective shown in FIG. 8A;

FIG. 9A shows a first example syringe assembly;

FIG. 9B shows a cross section view from the perspective shown in FIG. 9A;

FIG. 10 shows a cross section view of the syringe;

FIG. 11 shows a cross section view of a cable connector portion of the actuation assembly;

FIG. 12 shows a second example syringe assembly;

FIG. 13A shows a first partial parts exploded parts view of the second example syringe assembly;

FIG. 13B shows a second partial parts exploded parts view of the second example syringe assembly; and

FIG. 14 is a schematic of a computing system for controlling various operations of the cable driven syringe pump system.

The appended drawings are not necessarily to scale, and, at times, illustrate a simplified representation of various features illustrative of the principles of the disclosure. The specific design features of the present disclosure as described herein, including, for example, specific dimensions, orientations, locations, and shapes will be determined in part by the particular intended application and use environment. In the figures, reference numerals refer to the same or equivalent parts of the present disclosure throughout the several figures of the drawing.

### DETAILED DESCRIPTION

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The following discussion omits or only briefly describes certain components, features and functionality related to medical devices and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment or component has the same or nearly the same functionality or characteristic, unless the context or other statements clearly indicate otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Components of the disclosed embodiments can be fabricated from biologically acceptable materials suitable for medical applications, including metals, synthetic polymers, ceramics and bone material and/or their composites. For example, the components, individually or collectively, can be fabricated from materials such as stainless steel alloys, commercially pure titanium, titanium alloys, Grade 5 titanium, super-clastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, and their combinations.

Advantageously, the present disclosure is capable of providing a greatly improved system for administering drugs, especially during delicate surgical procedures during which high accuracy is essential. The system may include a syringe connected to and/or otherwise effectuated by a controller via a drive cable. The controller may be actuated by a variety of modalities, for example, a foot pedal, a switch, audio feedback, etc. In a conventional system, an actuator, e.g., a plunger, is often provided directly on the syringe which has the disadvantage of requiring a user to hold the syringe steady while simultaneously attempting to operate the actuator. Embodiments in accordance with the disclosure herein may improve operation accuracy by, e.g., providing the actuator remote from the syringe.

In various embodiments, a controller may be capable of being pre-programmed with relevant information, e.g., specific volume and flow rate information (of fluid or other materials) to be administered for a particular medicine and/or weight class of an individual. Medical treatments such as, e.g., ophthalmic drug therapies, may require a heightened precise control of the delivered dose and flow rate of the medication as a critical aspect of a successful therapeutic treatment plan. Another difficulty in these procedures may be the location of the medication delivery and the limited nature of the drug. For example, some medications such as stem cell and gene therapy treatments often require delivery beneath the retina of the eye (which is a very delicate surgical procedure requiring a steady injection, a precise dosage, and a precise delivery rate). In at least one relevant procedure, a surgeon may insert a cannula (or needle) into the eye, pierce the retina, and then deliver the injection beneath the retina. This procedure may require the surgeon to maintain perfect control of the cannula during the initial insertion and throughout the injection of material. Conventional systems that provide the actuator locally integrated with the syringe for manual operation by the surgeon substantially increase the risk of excessive movement of the cannula tip during the injection. For example, as the surgeon depresses the actuator movement of the cannula tip, which may be inside of delicate patient tissue, often occurs.

For these types of delicate procedures, other conventional systems have been developed that include an extension tube that connects the syringe to the cannula. However, these systems require a highly skilled assistant to actuate the syringe. These systems also still utilize manual operation (rather than automated operation) which increases the variability in injection pressure and in turn decreases the constant control of the injection. For example, manually depressing a plunger often leads to a non-constant delivery of material that may be in excess of a target delivery rate or below a target delivery rate and in some cases, over the total delivery time, both. In addition, a tubing between the injection site and the syringe requires the fluid to be filled in the dead space of the tube, thus increasing waste of the drug. Furthermore, the long tube may adversely affect the delivery due to fluid dynamics implications and friction of the sidewall of an elongated delivery tube vs. those fluid dynamics of a relatively smaller syringe. Other systems include a pneumatically driven syringe in which the injection pressure is set and controlled by a surgeon. However, both systems rely on manual reading of the graduation markings on the syringe which decreases the accuracy of the injection and specifically, increases the risk of inaccurate dose delivery. Conversely, embodiments in accordance with the present disclosure provide an improved system which provides accurate control of the dose delivery without manual operation of an actuator and without manual reading of the syringe graduation markings and with limited waste of the drug product.

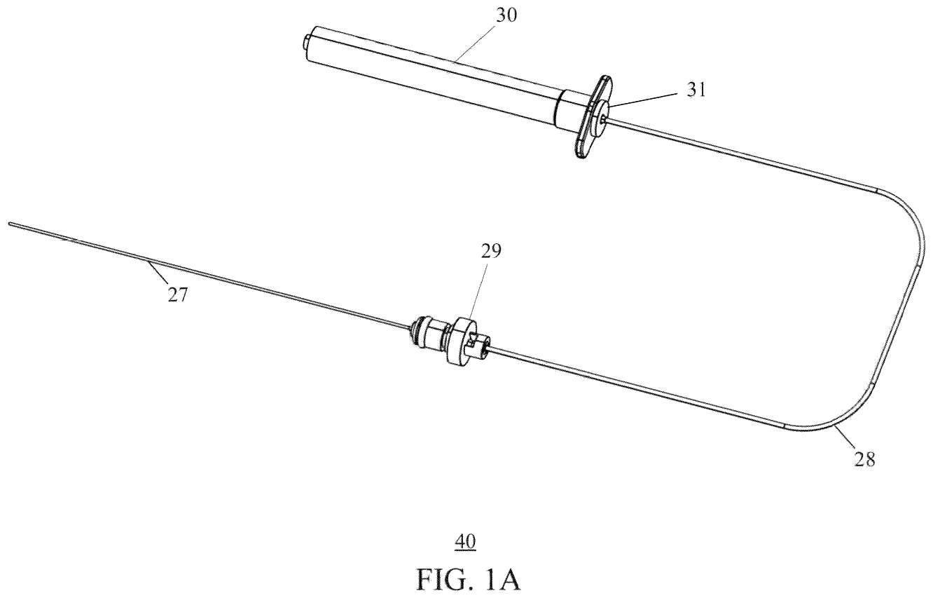

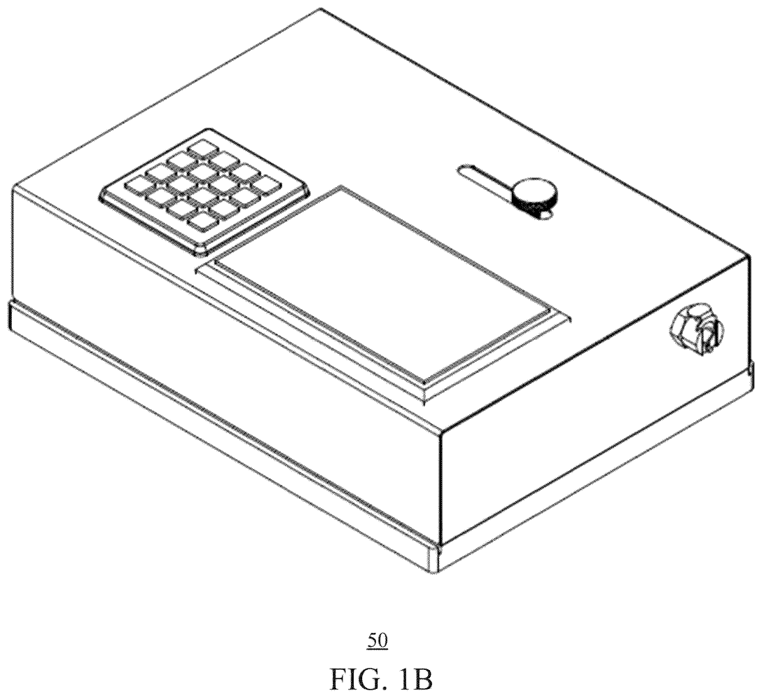

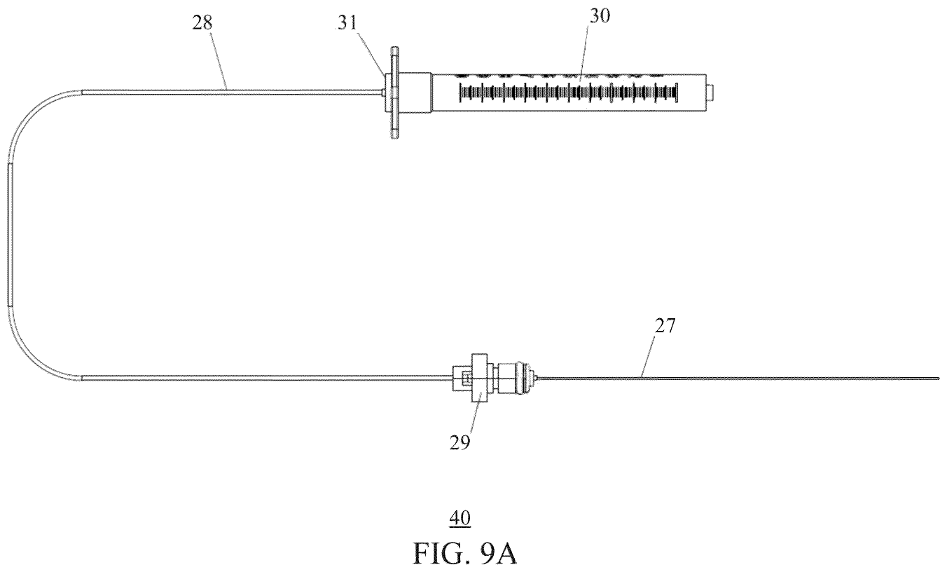

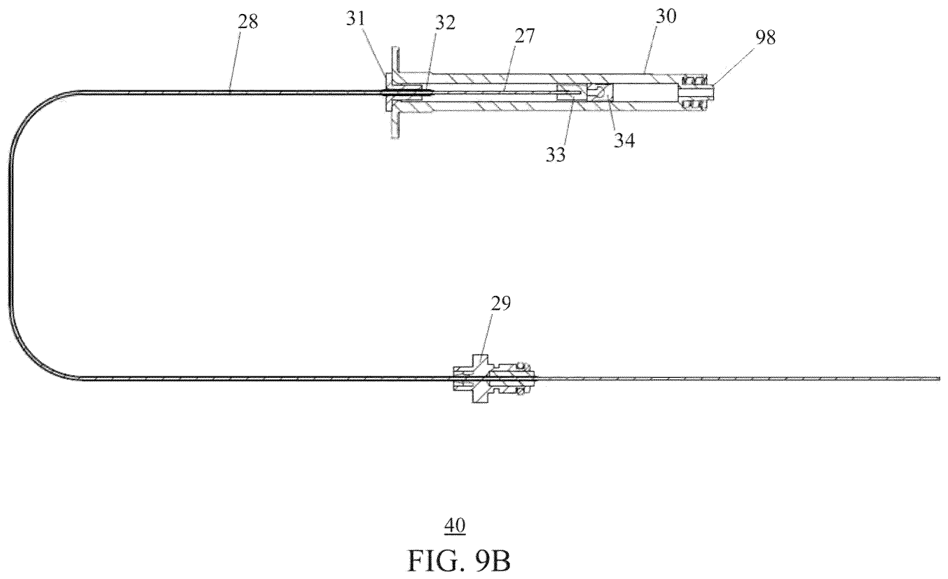

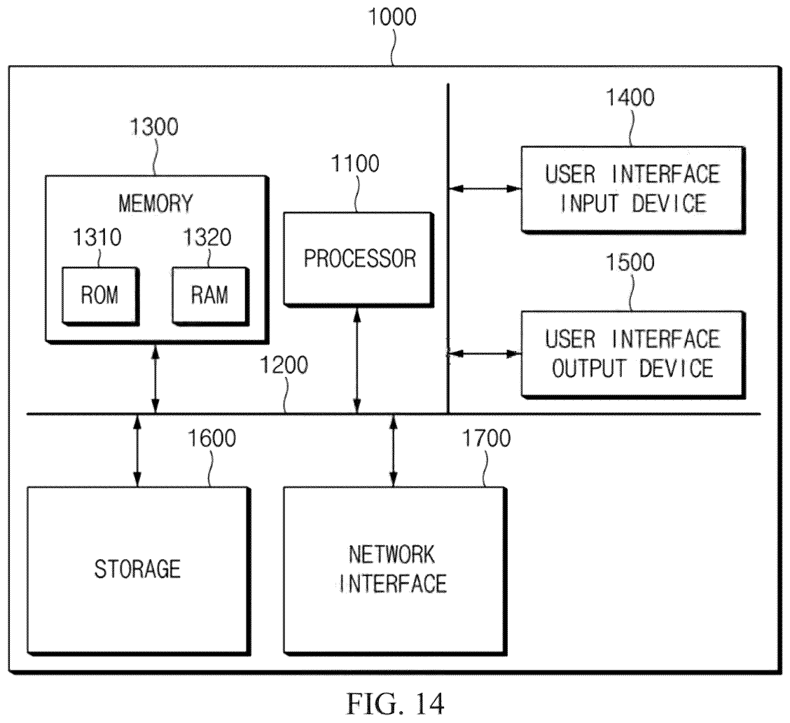

As shown in FIGS. 1A-1B, the present disclosure provides a system comprising two main components, the syringe assembly 40 and the control box 50. The syringe assembly 40 may include a syringe 30 and a drive cable that is connected to, e.g., the control box 50. In various embodiments, the syringe 30 may be integrally formed with the syringe assembly 40 or the syringe 30 may be configured to couple to and uncouple from a connector of the syringe assembly 40 as will be explained in further detail below with reference to the two embodiments shown in FIGS. 9A-13B. Additionally, the control box 50 may include various electronic components, such as a computing system 1000, as will be explained in further detail below with reference to FIG. 14. In various embodiments, the control box 50 includes an electrically operated actuator system that is controlled by a customizable software program containing computer executable code that when executed may cause the various components described herein to function, e.g., delivery of a precise dosage of material and at a precise flow rate.

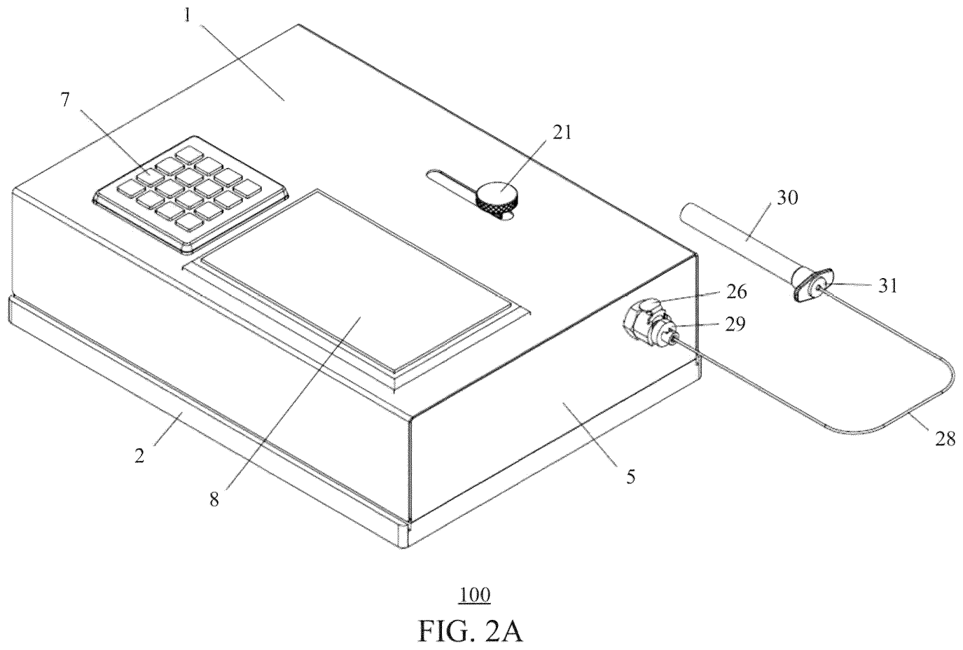

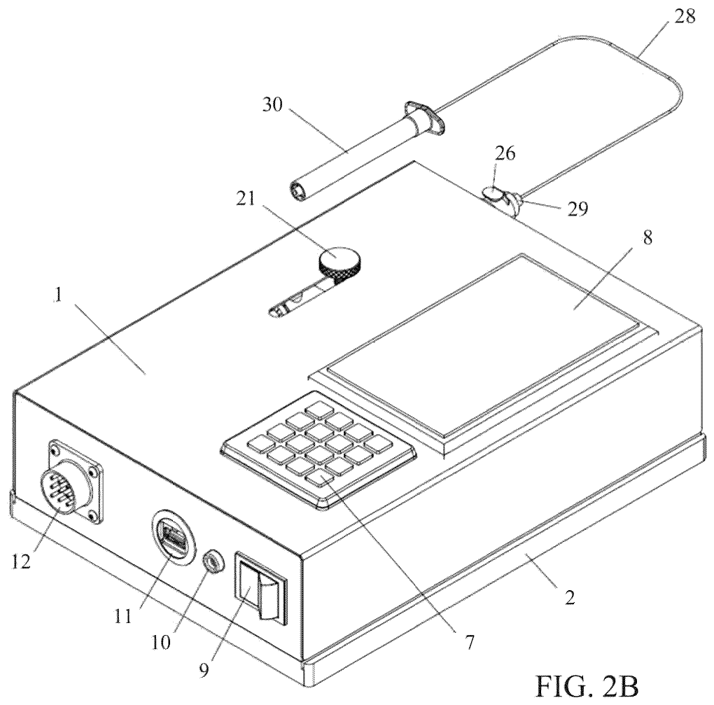

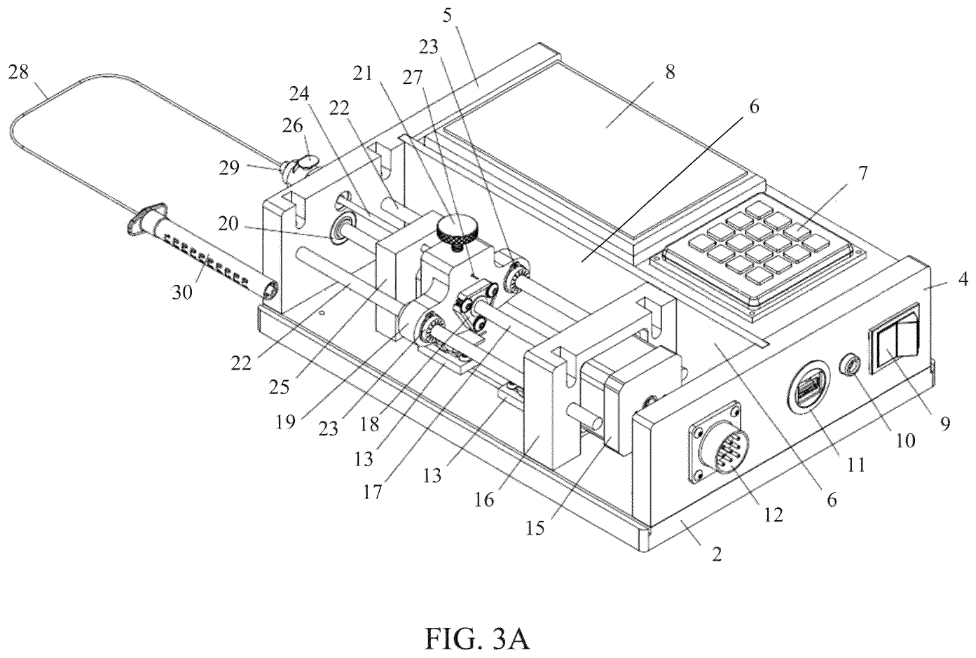

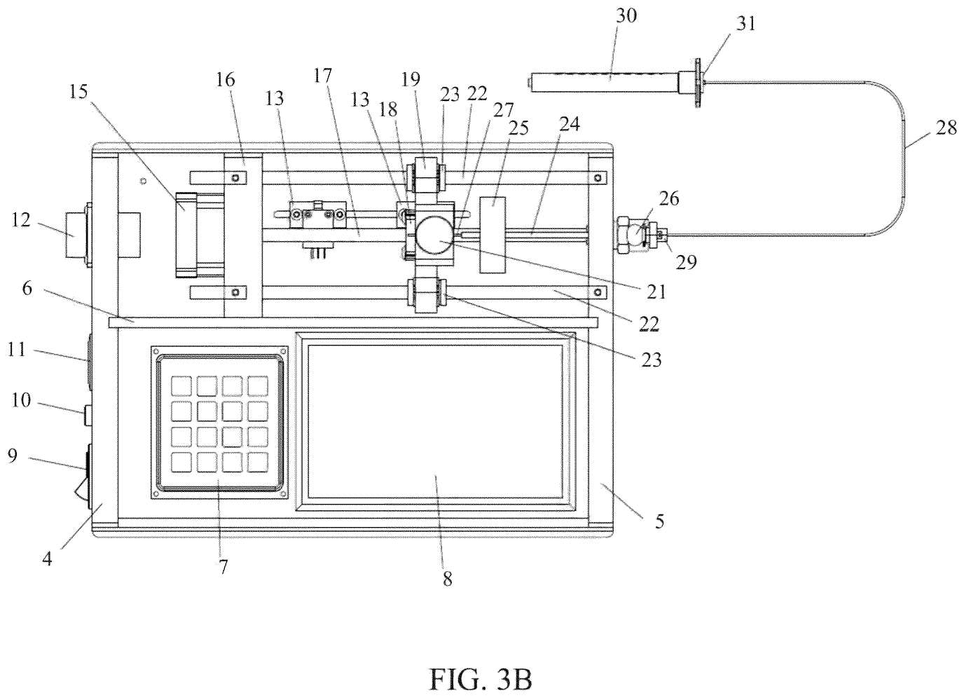

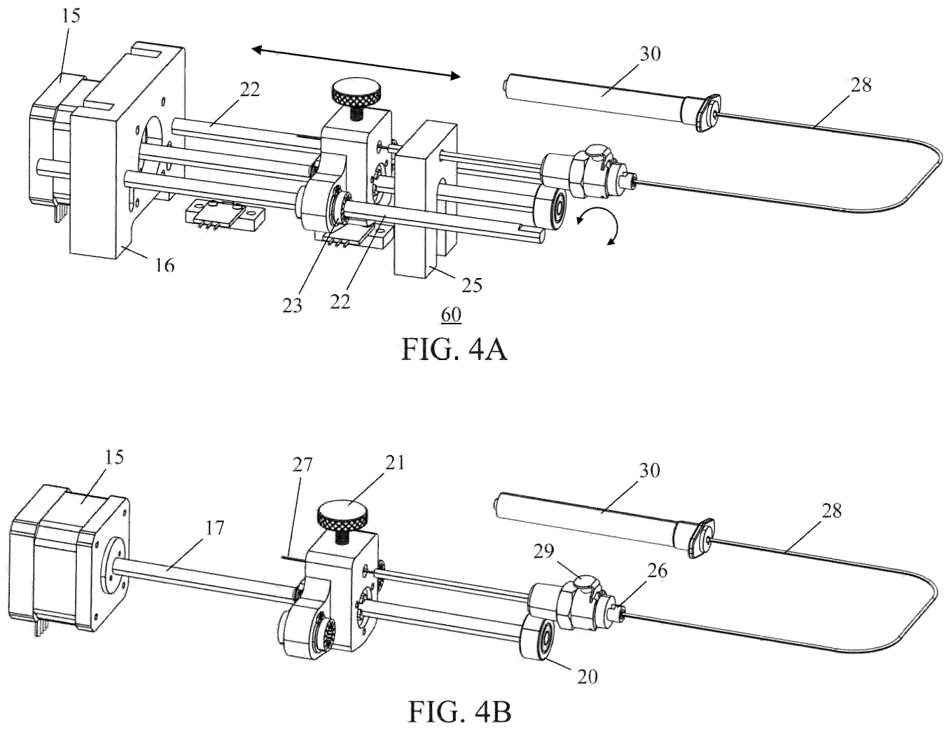

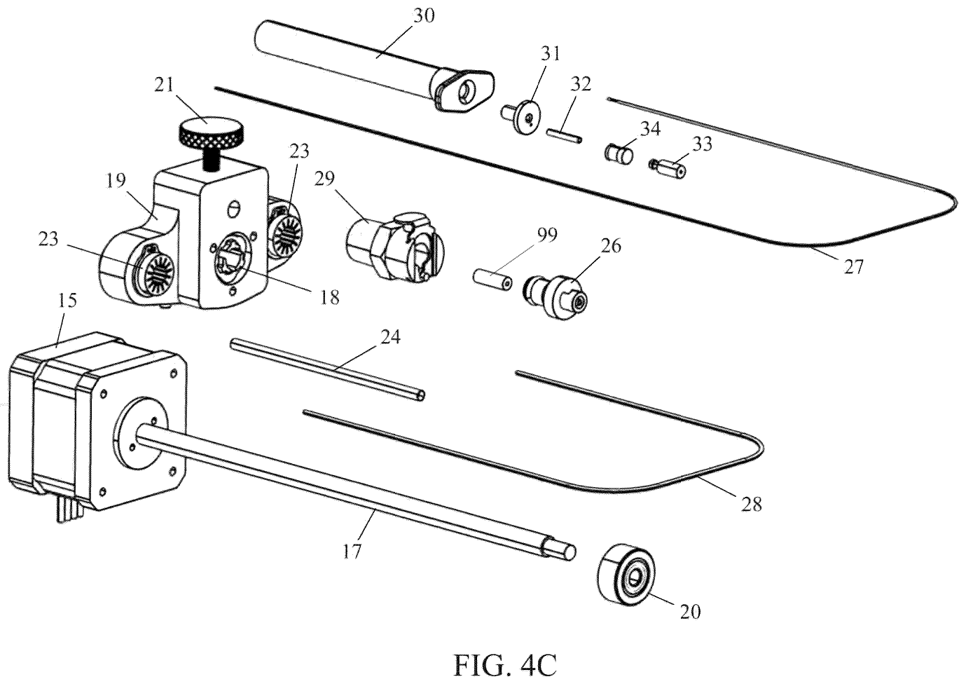

FIGS. 2A-2B show a first and second perspective view of the cable driven syringe system 100 in an assembled configuration. In particular, the control box 50 includes a housing formed by a cover 1, a base plate 2, a base foot 3, a switch/port side plate 4, a connector side plate 5, and an internal center plate 6 (see FIG. 3). The control box may further include digital input/output devices for receiving instructions from a user, e.g., a medical service professional, displaying an operating state to the user, and various ports for the user to control the delivery of a therapeutic material. By way of example, the control box 50 may include a keypad 7 (also referred to as a user interface input device), a display 8 (also referred to as a user interface output device), a power off/on switch 9, a power cable port 10 for receiving electrical power from an external source, a programming cable port 11 for receiving programmatic instructions from an external source, and a cable port 12 for controlling and/or initiating an automated delivery of a fluid or other therapeutic material. Also shown in FIGS. 2A-2B, the syringe 30 may include a rear cap 31 at a proximal end of the syringe which allows it to connect to a flexible sleeve 28, which functions similar to a protective sheath and/or outermost cannula that surrounds an interior flexible wire 27 (see FIG. 4C) as will be explained in further detail below. In turn, the flexible sleeve 28 may be connected to the control box 50 by inserting it inside of a central bore of sleeve connector 29 and a central bore of a control box connector 26. The sleeve connector 29 and control box connector 26 may be referred to collectively as a “connector,” and/or a “coupler” herein. In various embodiments, the control box connector 26 may include a thumb depress button for disconnecting the sleeve connector 29, the flexible sleeve 28, and/or wire 27. In disclosed embodiments, the flexible sleeve 28 and wire 27 collectively form a protected drive cable. For example, the flexible sleeve 28 and wire 27 may be connected to the control box 50 via the control box connector 26 and sleeve connector 29 to drive the delivery of material through the syringe 30. However, it shall be understood that in some embodiments only a single coupler, such as a quick connect coupler, is optionally utilized.

Referring generally to FIGS. 3A-4C, further details of the interior components of the control box 50, actuation assembly 60, and the syringe assembly 40 are illustrated. FIG. 3A illustrates a perspective view of some of the interior components of control box 50 and FIG. 3B illustrates interior components of the control box 50 from a top-down view. FIG. 4A shows an actuation assembly 60 which may be disposed inside of the control box 50 for causing the delivery of therapeutic material. FIG. 4B shows a partial parts view of the actuation assembly 60 and FIG. 4C shows an exploded parts view of the actuation assembly 60 with some parts removed for ease of understanding.

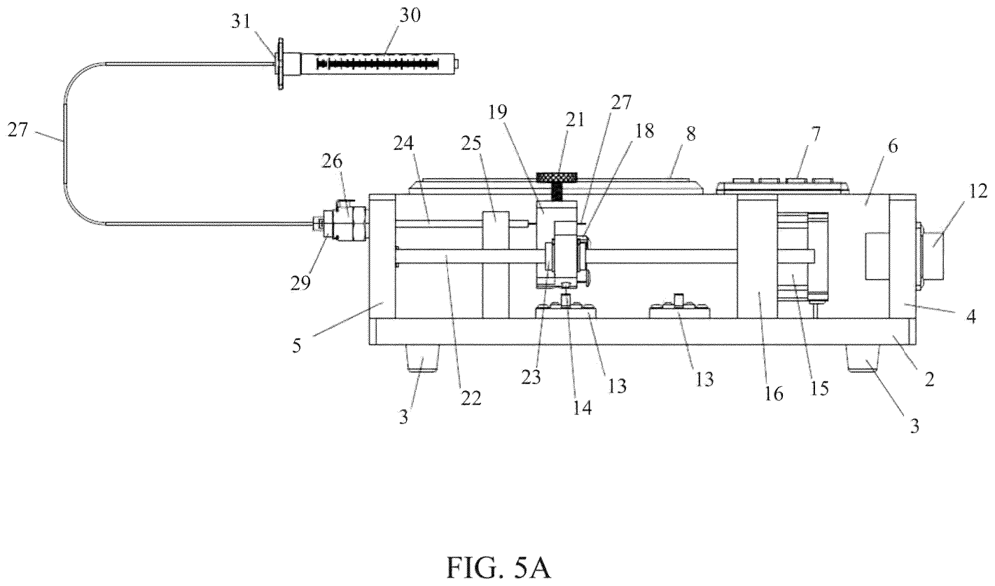

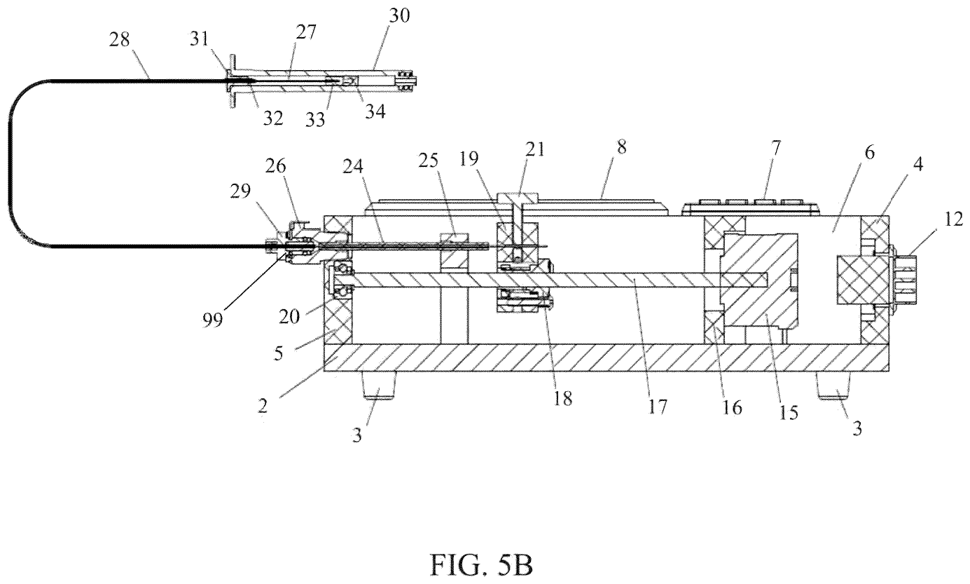

In various embodiments, the actuation assembly 60 may be disposed inside of the control box 50 in a first internal area of the housing defined by the switch/port side plate 4, internal center plate 6, and the connector side plate 5 (see FIGS. 3A and 5A). These components extend from the connector side plate 5 toward the switch/port side plate 4 and may be secured in place by any relevant supporting frame, structure, bolts, welds, etc. that are suitable. Moreover, the components in this first internal area form and/or function as an actuator system for operating and/or moving a stopper 34 (see FIG. 9B) within the syringe 30 in a precisely controlled way.

As mentioned above, the drive cable (flexible sleeve 28 and wire 27) connects to the control box 50 at the control box connector 26. Additionally, the drive cable may be surrounded by a connection segment 99 disposed within an internal portion of the control box connector 26 (sec FIGS. 5B, 7 and 13). In some embodiments, the flexible sleeve 28 portion of the drive cable may terminate at a location corresponding to connection segment 99 and the wire 27 may continue. For example, the wire 27 may then continue through a wire support tube 24, into a wire support mount block 25, and then terminate at a mounting block 19. In various embodiments, a user may easily couple the drive cable to the control box 50 by pushing the sleeve connector 29 against the control box connector 26. By doing so, the control box connector 26 may capture an end of the sleeve connector 29 while allowing the flexible wire 27 to extend through the bore of the control box connector 26. As explained above, the other end of flexible wire 27 may extend through rear cap 31. Rear cap 31 may be connected to and/or house sleeve support tube 32 in a central aperture thereof for supporting the sleeve 28 while allowing the flexible wire 27 to extend therethrough. In this embodiment, mounting block 19 may include an actuator such as a hand knob 21 to fasten and unfasten the flexible wire 27 (flexible inner wire) to a junction portion of the mounting block 19 in any feasible way. For example, with reference to FIG. 5B, the wire 27 may pass through an aperture of the threaded portion of hand knob 21, be coiled around a shaft of hand knob 21 (not illustrated), or may be pinched by the hand knob 21 against a support surface (not illustrated) or it may be otherwise immobilized somewhere downstream of the terminal end of the flexible sleeve 28 inside of the control box 50.

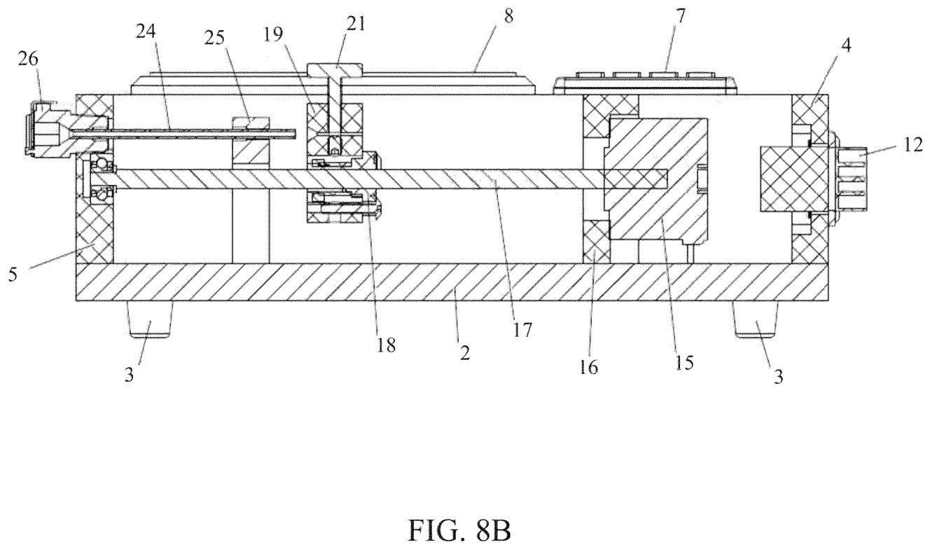

In terms of a supporting frame or structure for supporting the actuation assembly 60 within the control box 50, various beams, blocks, columns etc. may be provided as a counter torque for motor 15. In the illustrated embodiment two guide rods 22 and a shaft 17 extend from the connector side plate 5 through the mounting block 19 and slide bearings 23, and then they each extend through the motor mount block 16, which accommodates the motor 15. Motor 15 can be any type of electrical motor, e.g., a step motor may be advantageous because of the associated precision in mechanical movements relative to other motors. Additionally, in various embodiments at least a portion of shaft 17 may be threaded. For example, the shaft 17 may include a central threaded portion defined by a first thread pattern which may be engaged with a second corresponding thread pattern of mounting block 19. For example, as shown in FIG. 8A, mounting block 19 may include a bore that shaft 17 extends through. Shaft 17 may include a thread pattern that corresponds to a thread pattern of a threaded nut 18 that is fixedly coupled to the bore extending through mounting block 19. Alternatively, the bore itself may be threaded. In this way, shaft 17 and mounting block 19 may be threadably engaged such that rotation of shaft 17 by motor 15 causes linear translation of mounting block 19 in a direction parallel to shaft 17, as will be explained in further detail below.

With reference to FIG. 4A, when the motor 15 is actuated the shaft 17 rotates and transfers this rotational energy to the mounting block 19 which, in turn, translates this energy into a linear energy (due to being prevented from rotating) and thereby moving the threaded nut mounting block 19 forward and backward (shown by top double-sided arrow) along the shaft 17 while it rotates (shown by lower curved double sided arrow). In this way, the guide rods 22 slide through the slide bearings 23 and the flexible wire 27 is pulled and/or pushed in a forward and backward movement (shown by arrows) in a longitudinal direction of the shaft 17 of the motor 15. This forward and backward relative movement of mounting block 19 causes the flexible wire 27 to move forward and backward, thereby pulling and pushing a stopper 34 within the syringe 30 (see FIG. 9B), as described in further detail below. In various embodiments, it shall be understood that the choice of flexible wire 27 and braiding thereof may be chosen to ensure that flexible wire 27 has compression strength in an axial direction.

FIG. 5A illustrates a side view of the internal components of control box 50 and FIG. 5B illustrates a cross section view through a longitudinal axis of the shaft 17 of the motor 15. As shown in FIG. 5A, the travel limit switches 13 are mounted on the base plate 2 and each may interact with a travel limit magnet 14 mounted on the mounting block 19. As explained above, when the motor 15 is operated, the mounting block 19 moves forward and backward. In this configuration, the mounting block 19 may only move between the two travel limit switches 13 because the travel limit magnet 14 is attracted to each travel limit switch 13 and when it is close to a corresponding travel limit switch 13 the power of the motor (or the power supplied to the motor) is insufficient to overcome the relative attraction of the magnets 13, 14. In various embodiments, the magnets 13 and/or 14 may be electro magnets and/or permanent magnets. In other embodiments, a simple stop surface or catch may be utilized instead of the magnets 13, 14.

With reference to FIG. 5B, it shall be understood that the longitudinal extension direction of the shaft 17, mounting block 19, and wire support tube 24 collectively define the movement path of the specific portion of the flexible wire 27 that is located inside of the control box 50. Additionally, the portion the flexible wire 27 outside of the control box 50 is relatively unconstrained and can therefore move forward and backward within the flexible sleeve 28 at a multitude of relative orientations. In this sense, the motor 15 provides a very controlled forward and backward movement of mounting block 19 thereby providing a precise forward and backward movement of the flexible wire 27 and effectuating a precise delivery of a therapeutic material at a multitude of relative delivery angles. For example, as shown in FIG. 5B, a forward movement of mounting block 19 in the longitudinal direction of shaft 17 (towards left hand side of page) is about 180 degrees opposite from the forward movement of the stopper 34 (towards the right-hand side of page). This configuration thus provides a medical practitioner with great flexibility in the approach to a target surgical location.

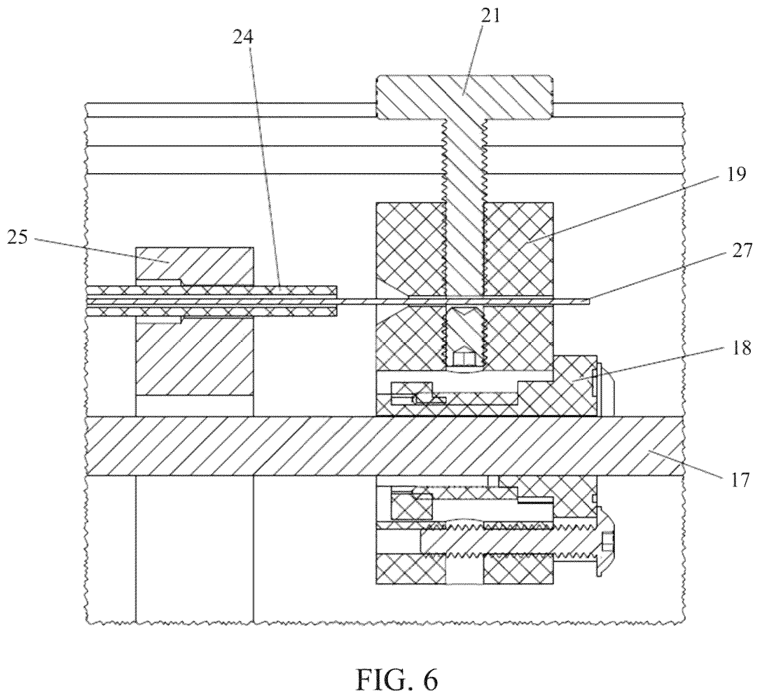

FIG. 6 illustrates an enlarged cross section view of a portion of the actuation assembly 60 where, from left to right, the flexible wire 27 is inserted through the wire support mount block 25, through the wire support tube 24, and into the mounting block 19. Once the flexible wire 27 has been inserted therethrough, the hand knob 21 may be turned to secure the positioning of flexible wire 27. In other words, the hand knob 21 anchors the flexible wire 27 portion of the drive cable to the actuation assembly 60 and thus allows the inner flexible wire 27 of the drive cable to move forward and backward as explained above.

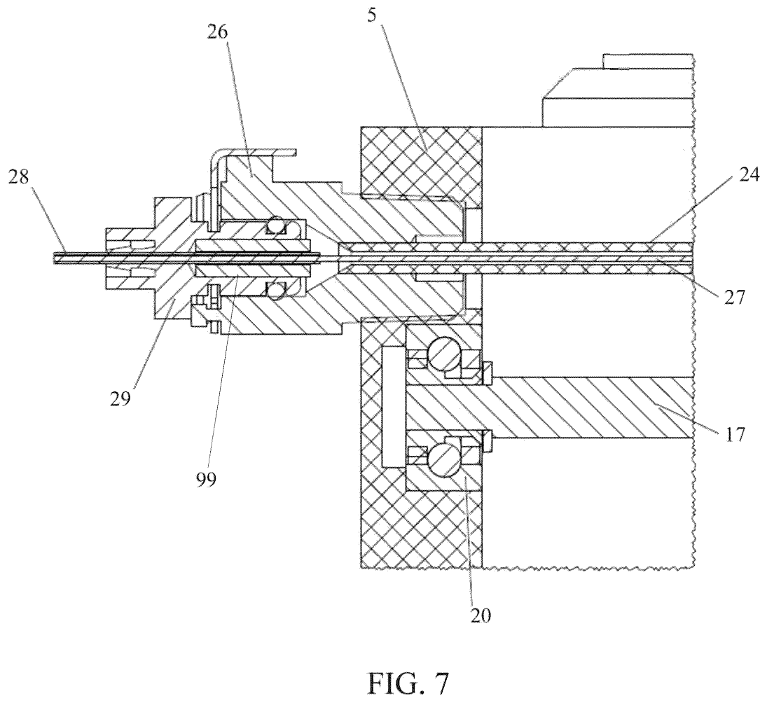

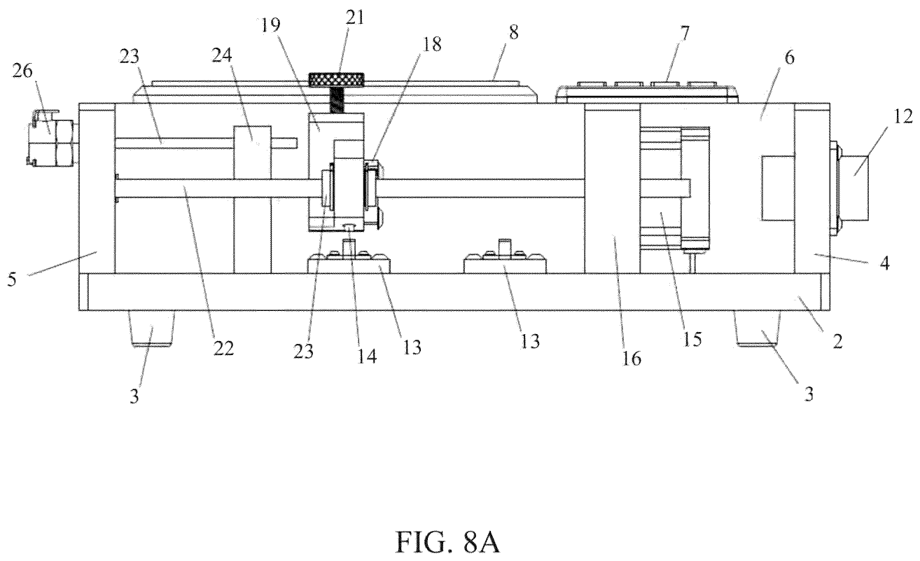

FIG. 7 illustrates an enlarged cross section view of the connection of the flexible wire 27 and flexible sleeve 28 at the junction in which they enter the control box 50. As shown, the flexible sleeve 28 of the drive cable surrounds the flexible wire 27. Each is inserted through the sleeve connector 29 and through the control box connector 26 and on through the side plate 5 via the wire support tube 24. In the region corresponding to the central bore of the control box connector 26, a connection segment 99 may surround the flexible wire 27 (which in turn surrounds flexible sleeve 28). On one end of the control box 50 the control box connector 26 is inserted into an aperture, or fitted groove, of the connector side plate 5 such that a central bore of the control box connector 26 may also accommodate the wire support tube 24 and the flexible wire 27 may extend therethrough. Also shown in FIG. 7, an end portion of the shaft 17 opposite from the motor 15 is secured to the side plate 5 by a ball bearing 20 that permits the shaft 17 to rotate in place.

FIGS. 8A-8B show a side view of the control box 50 with the components as described above. As shown, a cable port 12 is provided at the switch/port side plate 4. In one advantageous embodiment, this port may be configured to electrically couple to a foot control pedal such that an operator's hands are free to hold the syringe assembly 40 and the foot control pedal may effectuate the delivery of a therapeutic material. In various embodiments, the cable port 12 provides a connection to a user input device such as a remote-control mechanism for operating the motor 15 and/or other aspects of the control box 50. For example, the cable port 12 may provide a connection to a foot pedal, a joystick, a hand trigger, a keyboard, a rotatable knob, etc. that is operated by a user to activate the operation of the motor 15 and/or other aspects of the control box 50 based on the pre-programmed software. For example, the cable port 12 may be configured to receive an electrical signal from a user input device that is processed by a computing system 1000 of the control box (see FIG. 14). The electrical signal may include operation instructions that are processed by the computing system 1000 to perform any of the various mechanical functions via the actuation assembly 60, e.g., a forward motion, a backward motion, a forward motion at a specific rate or variable rate, a backward motion at a specific rate or variable rate, etc. Notably, the present disclosure is not limited to the use of the specifically listed user input devices and control mechanisms as other operational mechanisms are suitable and are contemplated.

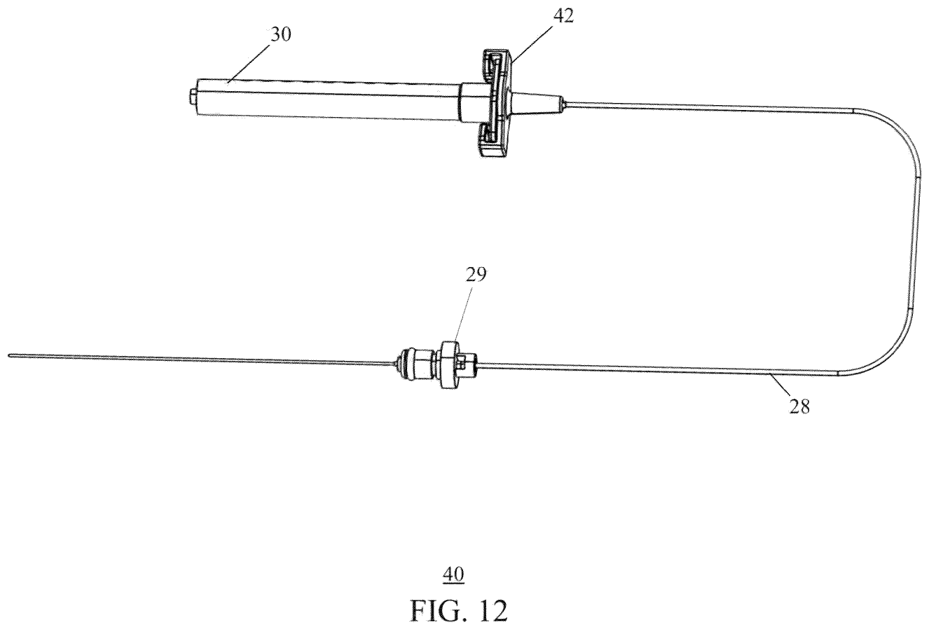

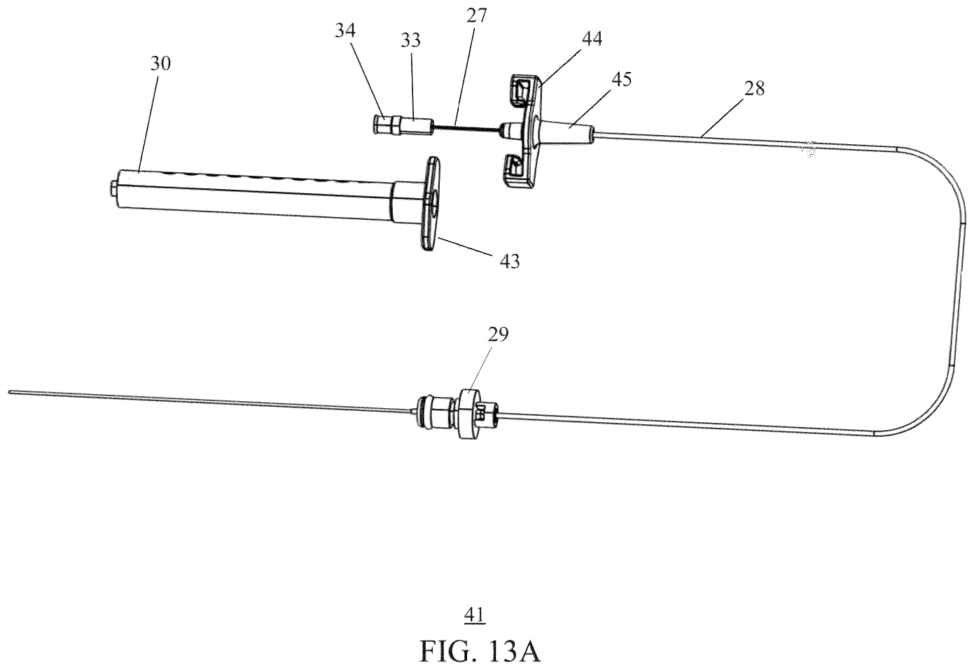

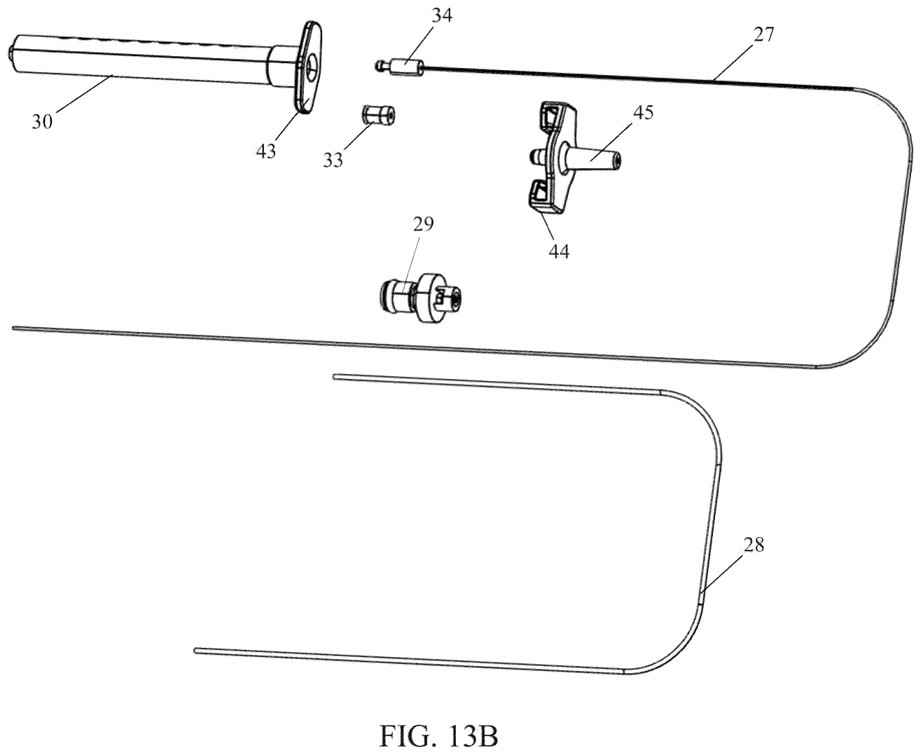

FIGS. 9A-11 show various views of a first embodiment of a syringe assembly 40 and FIGS. 12-13B show a second embodiment of a syringe subassembly 41. It shall be understood that the first embodiment and second embodiment of the syringe assemblies 40, 41, respectively, may include the same, similar, and/or substantially the same components and functionality as one another. Accordingly, duplicative description will be omitted and like parts will be used to the extent possible.

Referring generally to FIGS. 9A-12, the syringe assembly 40 may include the syringe 30 with a tip 98 at a distal end thereof for connecting to a cannula (first end) and a rear cap 31 at a proximal end (second end) thereof. In various embodiments, the tip 98 may connect to and be unconnected from a cannula, needle or other aspiration tube as would be understood by a person of ordinary skill in the art. Additionally, in at least some embodiments an occlusion sensor may be disposed in the syringe and be configured to detect if the syringe is occluded, e.g., if a fluid is not properly being delivered. In some embodiments, the system 100 can detect if there is an occlusion by detecting a feedback force through the cable 27, e.g., with a stress or strain gauge. In other embodiments, a flow rate sensor may be disposed within the syringe. For example, the flow rate sensor may be configured to detect a volume and flow rate of fluid being dispensed from the syringe.

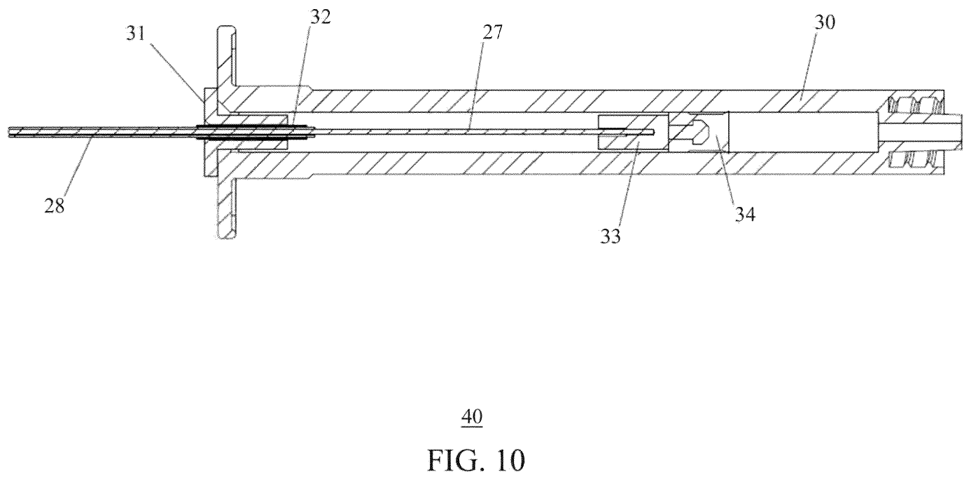

As seen best in FIG. 9B and FIG. 10, the rear cap 31 includes a central bore that provides an entry point for connecting the flexible sleeve 28 and inner wire 27 of the drive cable to the syringe 30. As illustrated, only the flexible wire extends through the central cavity of the syringe 30 and is attached to a wire end adapter 33. In various embodiments, the wire end adapter 33 may include a protrusion that in turn is coupled to a corresponding cavity in the stopper 34. For example, a bulbous mushroom end of the adapter 33 is coupled to a corresponding receiving aperture of the stopper 34 within the syringe 30. The stopper 34 is constrained from lateral movement due to its corresponding shape with the interior sidewalls of the syringe 30 while permitting forward and backward motion in a longitudinal direction of the syringe 30 by permitting the stopper 34 to slide within the interior of the syringe 30 based on the movement of the wire 27 as the actuator is operated. In this way, the wire 27, adapter 33, and stopper 34 function in a similar manner as a plunger of a traditional syringe.

As explained previously, the drive cable comprises the flexible sleeve 28 in which the flexible wire 27 is accommodated therein such that the flexible wire may slide forward and backward within the flexible sleeve 28. In various embodiments, the drive cable may be formed to provide a minimal gap of about 0.001 inches to about 0.002 inches between the flexible outer sleeve 28 and the flexible inner wire 27. For example, an outer diameter of the flexible wire 27 and an inner diameter of the flexible sleeve 28 may provide a minimal gap to prevent air entrainment and contamination and generally provide a lightweight comfortable to use device. In various embodiments, the flexible wire 27 may have a thickness of about 0.025 inches to about 0.027 inches and the overall thickness of the drive cable may be about 0.050 inches to about 0.052 inches. However, it shall be understood these dimensions are merely exemplary and of course the wire 27 and sleeve 28 may even have varying thickness and supporting intermediate sleeves or bushings across a length thereof. However, it shall also be understood that a relatively thin diameter of the drive cable 27 has certain advantages. In various embodiments, the flexible sleeve 28 may include a reinforcing wire braid around an outside thereof that protects a user of the device while also eliminating and/or suppressing axial and lateral expansion of the flexible sleeve 28, e.g., bulging. Additionally, an inner lumen of the flexible sleeve 28 may include a polytetrafluoroethylene (PTFE) layer that reduces the friction between the flexible sleeve 28 and the flexible wire 27 during movement. In this sense, a flexible sleeve 28 having low friction interior sidewalls and a relatively small gap between the low friction sidewalls and the inner wire 27 may be especially advantageous for effectuating the linear motion of the actuation assembly 60 into linear motion of the wire 27. In various embodiments, the flexible sleeve 28 may be made of, e.g., a polyimide material and the flexible wire 27 may be made of a nitinol material. However, the present disclosure is not limited to these materials. Additionally, the cable may have a length of about 24 inches to about 36 inches which allows for sufficient separation between the syringe and the control box 50.

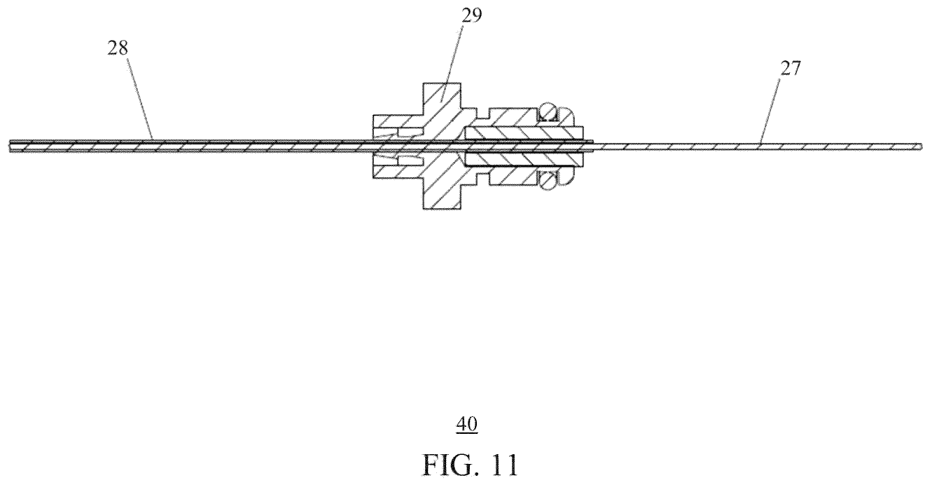

FIG. 11 is an enlarged view of the sleeve connector 29 and the control box connector 26. As mentioned previously, the first end of the drive cable may be attached to the syringe 30 and the second end of the drive cable may be releasably attached to the actuation assembly 60 via the connector 29 and control box connector 26. As also shown in FIG. 11, the flexible sleeve 28 and wire 27 are both coupled to the connector 29 and control box connector 26 but only the flexible wire 27 extends deep into the control box 50 for coupling to the actuation assembly 60. For example, the flexible wire 27 may extend about 6 inches past the second end of the flexible sleeve 28 and into the control box 50.

Referring to FIGS. 12-13B generally, a second example syringe subassembly 41 is shown. In this embodiment, the syringe 30 is connectable to and detachable from a connector 42. The connector 42 may take any form such as a quick connect pop on style connector or as a connector in which a connecting flange 43 of syringe 30 may be disposed inside of a cavity formed by a corresponding winged double flange 44 connected to the drive cable. In this way, the proximal end of syringe 30 may include a flange 43 that may be rotated into place within a cavity defined by the winged double flange 44 of the drive cable. As seen best in the exploded parts view of FIG. 13, the wire 27 may extend beyond the winged double flange 44 and into the syringe 30. For example, the wire end adapter 34 may be permanently affixed to an end of the wire 27 to such that a protrusion of the adapter 34 may be removably connect to a correspond aperture of the stopper 34 as explained previously. Additionally, in various embodiments, the winged double flange 44 may be connected to or integrally formed with a reinforcing structure 45 to securely couple to the flexible sleeve 28. In this example, the reinforcing structure 45 is integrally formed with the winged double flange 44 and includes a central bore therein permitting the flexible wire 27 to pass therethrough (see FIG. 13B). In at least some embodiments, the reinforcing structure 45 may include an outer ring and a deformable inner ring that are rotatable relative to one another to provide a clamping force by decreasing a size of an internal diameter of the bore extending through a slotted inner ring portion. For example, an end user may rotate the outer ring towards the end of the reinforcing structure 45 thereby radially compressing the sidewalls of the slotted inner ring and providing a clamping force thereto for securing the drive cable. This configuration may be advantageous for coupling the winged double flange 44 to and from the flexible sleeve 28. In this sense, it may be advantageous to allow for most, many, or even all of the parts of the syringe subassembly 41 to be able to be disconnected to service and/or sterilize the various parts between intermittent uses and/or prevent the cross contamination of therapeutic materials.

FIG. 14 is a schematic of a computing system 1000 that may be housed in the control box 50. In at least one embodiment, the computing system 1000 is disposed in an opposite cavity and on an opposite side of the internal center plate 6 from the actuation assembly 60 (see FIG. 3A). This relative location may place the computing system 1000 directly underneath the keypad 7 and display 8. Referring to FIG. 14, a computing system 1000 may include at least one processor 1100, a memory 1300, at least one user interface input device 1400, at least one user interface output device 1500, a storage 1600, and an optional network interface 1700. These electrical components may, in turn, be in electrical communication via a bus 1200. It shall be understood that the keypad 7 may be one of many applicable user interface input devices 1400 and that the display 8 may be one of many applicable user interface output devices 1500.

In various embodiments, the processor 1100 may be a central processing unit (CPU) or a semiconductor device that processes instructions stored in the memory 1300 and/or the storage memory 1600. The memory 1300 and the storage 1600 may include various volatile or nonvolatile storage media. For example, the memory 1300 may include a read only memory (ROM) and a random access memory (RAM) which may also be referred to as a “non-transitory” memory as would be understood by a person of ordinary skill in the art. In particular, the processor 1100 and/or computing system 1000 at large may be referred to as a “controller” and it may be configured to send and receive various electric signals which in turn are outputted to various mechanical devices to effectuate the delivery of a therapeutic material. For example, the computing system 1000 may be configured to control the motor 15 by the sending and receiving of electrical signals (i.e., a current amount or power) to effectively deliver a precise and highly accurate dosage amount of a therapeutic material and at a specific predetermined flow rate.

Accordingly, methods of operating the cable driven syringe system 100 may be implemented directly by hardware executed by the processor 1100, a software module, or any combination thereof. The software module may reside in a storage medium (that is, the memory 1300 and/or the storage 1600), such as a RAM memory, a flash memory, a ROM memory, an EPROM memory, an EEPROM memory, a register, a hard disk, a detachable disk, or a CD-ROM. The example storage medium may be coupled to the processor 1100, and the processor 1100 may read information from the storage medium and may write information to the storage medium. In another example, the storage medium may be directly integrated with the processor 1100. The processor and the storage medium may reside in an application specific integrated circuit (ASIC) as well. In this embodiment, the ASIC may reside in a user terminal, e.g., the control box 50.

In various embodiments, the software module may include various types of information specific to a particular type of therapeutic material. For example, a first therapeutic material may be delivered for a dosage of 20 microliters and at a flow rate of 5 microliters per second. A second therapeutic material may be delivered for a dosage of 40 microliters and at a flow rate of 20 microliters per second. In this sense, the computing system 1000 may have predetermined information to effectively deliver a dosage of a specific material in view of best practices in the industry. Furthermore, the computing system 1000 may be updated periodically from time to time via, e.g., the network interface 1700. Additionally, network interface 1700 may be configured to send and transmit operating information to an external server owned and operated by the manufacturer or a relevant third party. In this sense, the manufacturer may receive relevant operating information of the cable driven syringe system 100 and may also be able to notify an owner or operator of the cable driven syringe system 100 when maintenance may be required.

Furthermore, through extensive testing performed by the manufacturer, the software may be pre-programmed to know precisely how much power to send to the motor 15. In this sense, the particular material attributes of the wire 27, flexible sleeve 28, the length thereof, the internal friction thereof, the material attributes of the stopper 34 etc. affect how much power should be transmit to the motor 15 in view of a specific dosage and flow rate target requirements for a particular therapeutic material. In at least one embodiment, the software is programmed to ensure that a delivery of a therapeutic material is delivered with an accuracy of about +/−3 microliters by precisely controlling the amount of power transmit to the motor. For example, the computing system 1000 may be configured to control a power of an electrical control signal transmit to the motor such that a delivery of a volume of a therapeutic material is performed with an accuracy of about +/−3 microliters.

With the attributes of the cable driven syringe system 100 now being fully enabled, the operation of the system components will be described by referring back to the various numbering scheme shown in the FIGS. when convenient. Prior to use of the cable driven syringe system 100, the syringe 30 may be pre-filled with a fluid or other therapeutic material. Alternately, prior to injecting an end of a cannula connected to the syringe 30 into the injection site, the fluid may be loaded into the syringe 30 by operating the actuation assembly 60 to retract the stopper 34 and aspirate the fluid into the syringe 30. Then, the actuator assembly 60 may be operated to slowly advance the stopper 34 within the syringe 30 to purge any air bubbles within the syringe 30 and the cannula tip connected thereto. Either prior to filling the syringe 30 or thereafter, a user may enter desired dose parameters including volume and flow rate using the keypad 7 on the control box 50. The display 8 may show a confirmation of the entered parameters. The display 8 may also be used to output various notifications to the user such as a start notification and a stop notification of the fluid dispensing, start and stop of the actuation assembly 60, a remaining dose amount to be administrated, a counter, or any other contemplated visual notifications. The dose parameters may also be stored in an operation software program stored within a memory storage 1300, 1600 of the control box 50 for a specific type of therapeutic material as explained hereinabove.

Once the syringe 30 is filled, the syringe 30 may be connected to the drive cable including the flexible wire 27 and the flexible sleeve 28. The drive cable may then be connected to the actuator assembly 60 as described above. A cannula tip attached to a distal end of the syringe 30 may then be inserted into the injection site. For example, the cannula may be inserted into the eye and the tip thereof placed beneath the retina. Once the cannula tip has been placed at an injection site, the syringe 30 may be operated using the actuator. For example, the motor 15 may be actuated on/off by using a foot pedal connected to port 12 which is operated by a first medical practitioner. Alternatively, a button may be manipulated by a second medical practitioner to begin the process of dispensing a predetermined desired dose volume and flow rate of the fluid while the first medical practitioner holds the syringe 30 steady. The discharging or dispensing of the fluid stored within the syringe 30 into the injection site may be based on a stored operation program as explained above. In particular, the actuation of the motor 15 may cause a forward movement activation of the stopper 34 to move within the syringe 30 based on the push and pull movement of the flexible wire 27 as discussed above.

As another example, a user may elect to dose 50 microliters of fluid at a speed of 10 microliters per second. When ready to deliver the predetermined dose, the user may manipulate the foot pedal or button to initiate the operation software program. This process eliminates the need for the user to manually control the dosage or even visually confirm a predetermined dosage since the program controls both the amount of fluid to be administered as well as the flow rate at which it is discharged. The discharge of the fluid may be stopped automatically once the operation program is complete and optional sound or visual indication may be provided to indicate that the procedure has been completed. For example, the discharge of the fluid may be stopped when the volume of the fluid reaches a predetermined threshold. After the operation has been initiated, the program may also be stopped and restarted at any time by the user. This may be accomplished by, for example, disengaging the foot pedal or pressing the button again. The present disclosure, however, is not limited to such manipulations to stop the program.

Advantageously, the cable driven syringe pump system 100 described herein addresses limitations of conventional systems and challenges of particular drug therapies using a system that provides precise administration of medications during delicate surgical procedures requiring extremely small doses of a drug. The system 100 described herein allows the syringe containing the fluid to be administered to be located at the injection site thus preventing waste of the drug product typically remaining in conventional system with relatively long tubes that are delivered by fluid mechanisms and pumps. That is, typically, the dead space in the tubing must be filled with the fluid prior to the injection which unnecessarily consumes the drug. In addition, the system includes a control box 50 with an actuation subassembly separated from the syringe 30. This may eliminate and/or greatly suppress added potential vibration introduced from a medical practitioners hands and also decreases the weight a surgeon must bear during the performance of various procedures. In this way, the system reduces unintended movement of a cannula tip attached to syringe 30 during the injection process and also provides an ergonomic configuration improving user comfort.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

1. An injection system, comprising:
a syringe into which a fluid is filled and from which the fluid is discharged to an injection site;
a stopper coupled to and slidable within an interior of the syringe;
an actuator configured to control the stopper within the syringe, wherein the syringe is located remote from the actuator; and
a drive cable connecting the actuator and the syringe,
wherein the drive cable includes:
a flexible inner wire; and
a flexible outer sleeve enclosing the flexible inner wire.

2. The injection system of claim 1, wherein a first end of the flexible inner wire is connected to the stopper and a second end of the flexible inner wire is configured to be releasably connected to the actuator.

3. The injection system of claim 1, wherein a minimal gap is formed between the flexible inner wire and the flexible outer sleeve.

4. The injection system of claim 3, wherein the minimal gap is about 0.001 to 0.002 inches.

5. The injection system of claim 1, wherein the flexible outer sleeve includes a reinforcing wire braid configured to suppress axial and lateral expansion of the flexible outer sleeve.

6. The injection system of claim 1, wherein the flexible outer sleeve comprises a polyimide material.

7. The injection system of claim 1, wherein the flexible inner wire comprises a nitinol material.

8. The injection system of claim 1, wherein the flexible inner wire has a thickness of about 0.025 inches to about 0.027 inches.

9. The injection system of claim 1, wherein an inner lumen of the flexible outer sleeve includes a polytetrafluoroethylene (PTFE) layer configured to reduce friction between the flexible inner wire and the flexible outer sleeve during movement of the flexible inner wire.

10. The injection system of claim 1, wherein the syringe is releasably coupled to the drive cable.

11. The injection system of claim 1, wherein the actuator is connected to a controller configured to execute an operation program of the injection system.

12-13. (canceled)

14. The injection system of claim 1, wherein the actuator is actuated by a foot pedal or a button.

15. (canceled)

16. The injection system of claim 1, wherein the syringe is configured to connect to a cannula tip through which the fluid is discharged into the injection site.

17. (canceled)

18. A method of injecting a fluid to an injection site, comprising:
connecting a syringe to a drive cable, wherein the drive cable includes a flexible inner wire and a flexible outer sleeve enclosing the flexible inner wire;
connecting the drive cable to an actuator connected to a controller;
injecting a cannula tip connected to the syringe into the injection site;
receiving, at the controller, an operation program; and
actuating the actuator to activate a stopper coupled to and slideable within an interior of the syringe based on the operation program to discharge a fluid stored within the syringe into the injection site,
wherein in the actuating of the actuator, the actuator is moved forward and backwards to push and pull the flexible inner wire and move the stopper within the syringe.

19. The method of claim 18, wherein a first end of the flexible inner wire is connected to the stopper and a second end of the flexible inner wire is releasably connected to the actuator.

20. The method of claim 18, wherein the operation program includes a volume and a flow rate of the fluid to be discharged from the syringe.

21. (canceled)

22. The method of claim 18, wherein the syringe is pre-filled with the fluid.

23. The method of claim 18, further comprising:
prior to injecting the cannula tip connected to the syringe into the injection site, operating the actuator to advance the stopper to purge air bubbles within the syringe and/or the cannula tip.

24. The method of claim 18, wherein the actuator is actuated via a foot pedal or a button.

25. The method of claim 24, wherein the foot pedal or the button are located remote from the syringe.

26. The method of claim 18, wherein the discharge of the fluid is stopped automatically in response to determining that the operation program is complete.

27. The method of claim 18, wherein the discharge of the fluid is stopped when the volume of the fluid reaches a predetermined threshold.

28. (canceled)

29. A cable driven injection system, comprising:
a syringe including a stopper disposed within an interior cavity thereof, the syringe being configured to receive a fluid and discharge the fluid at a surgical site;
an actuator assembly including an electric motor and a rotatable shaft configured to linearly translate a block in a longitudinal direction;
a drive wire coupled to the block and the stopper, the drive wire being configured to move forward upon activation of the actuator assembly thereby moving the stopper forward and causing the syringe to discharge the fluid; and
a control box having a computing system including at least one controller and a non-transitory memory storage containing an operation program comprising computer executable code, the operation program containing target volume information for the discharge of the fluid,
wherein the controller is configured to supply a target power to the electric motor such that a volume of fluid discharged from the syringe corresponds to the target volume information of the operation program.

30-37. (canceled)
